(19) United States
(12) Patent Application Publication
(10) Pub. No.: US 2018/0147327 A1
(43) Pub. Date: May 31, 2018

(54) Printed article having a cellulose thermoplastic alloy composition and molding thereof

(71) Applicant: Robert C. Joyce, Toledo, OH (US)

(72) Inventor: Robert C Joyce, Toledo, OH (US)

(21) Appl. No.: 15/449,795

(22) Filed: Mar. 3, 2017

Related U.S. Application Data
(60) Provisional application No. 62/497,624, filed on Nov. 28, 2016.

(51) Int. Cl. A61L 31/04; A61L 31/08; A61L 31/14; B33Y 80/00; B33Y 70/00; B01D 71/10; B01D 71/56; B01D 71/26; B29C 64/112

(52) U.S. Cl. CPC A61L 31/042; A61L 31/082; A61L 31/041; A61L 31/146; B33Y 80/00; B29K 2023/12; B01D 71/10; B01D 71/56; B01D 71/26; B29C 64/112; B33Y 70/00

(57) ABSTRACT

A molded article consisting of an organic compound, preferably cellulose fibers having a glass transition point above 200 C and melt blended with thermoplastic polymers of different melt temperatures, whereby, a 3 or 4 Dimensional printer that may consist of an additional 5 or 6 axis, has cellulose fiber particles that are layered and compressible in a semi solid state. These layers having non oriented cellulose fiber particles will help produce a buoyant molded article with a surface texture that can absorb moisture and attract gases for coatings.

### CROSS REFERENCES

Application claims priority of U.S. Provisional Patent Application Ser. No. 62/497,624 filed Nov. 28, 2016 this prior application are incorporated herein by reference.

### FIELD OF INVENTION

The present invention is a molded article produced from a 3 or 4 dimensional printing process, utilizes an advanced biopolymer material composition that includes an organic compound, i.e. cellulose fibers with hydrophilic and hydrophobic polymers, whereby, when printed the cellulose fiber particles are layered and non orientated semi solid state. These particles are not Compressed to help form layers that are overlapping to help trap specialized fluids or gases, and after solidification, the molded article will be buoyant, having a specific gravity less than ASTM or ISO methods to establish specific gravity of the biopolymer. These molded articles have many applications, especially in replacing some existing medical filters that consisted of materials such as polymers and synthetics. These medical filters would consist of cellulose, more specifically cellulose nitrate and cellulose acetate, nylon, polypropylene and plotter-fluoroethylene (PTFE) Some of these filters require adhesives with heat or other types of secondary processes to form these molded articles. i.e. filters, membranes. Commercially available membranes are made from a single polymer and composite membranes will incorporate a backing material to increase physical integrity. PTFE membranes require the added strength of a polypropylene or polyethylene backing for easy handling. Hydrophobic borders like polypropylene and polyethylene can help prevent spillover. Membranes can come in flat sheet or assembled cartridges. Also, some membranes can be dyed different colors or have grid lines added for cell counting which will require different types of pigments. In existing filter construction, multiple materials can complicate designs, effect quality of the construction and performance. Even one material, such as metal can cause complications if implanted into a human body.

Molded articles have consisted of one or more polymers, and in some cases, are not adequate to achieve the optimum performance desired in the application. Improved performance by combining polymers has evolved and produced some unique molded articles. These molded articles are produced from processes, i.e. injection molding, extrusion, micro molding that require molding pressures to inject the polymers, with or without fillers into the mold. A polymer with a filler like natural fibers, can improve compressibility, which can dramatically change the performance of the molded article. By combining polymers or alloying thermoplastic resins with natural fillers. i.e. wood flour, the molded articles can also have an increase in strength and flexural properties. Previous technologies utilizing cellulose fibers that include microbial cellulose with multiple thermoplastics for molding articles have focused on the compression of the fibers for increasing tensile strength and flexural modulus. These processes will compress the fibers to the point where a richer polymeric surface is evident, and the cellulose fiber is compressed. Conversely, without high pressures in a process like 3 D printing, there will be a loss in properties and density, however, based on the inventors discovery tiny cellulose fiber particles having a hydrophilic and hydrophobic coating with increased adhesion can produce a unique molded article.

A printed article produced by a 3 D printing process with a compound that includes hydrophilic and hydrophobic polymers with an organic compound, i.e. cellulose fiber can alleviate complicated designs and improve performance in applications. The inventor has identified two compounds to form one composition, a cellulose thermoplastic alloy composite, that will specifically utilize polyamide, polyolefin, cellulose fiber, compatibilizer, modified elastomer and moisture. This composition has been further processed in layers produced in a printing process to achieve qualities such as absorbency, biocompatibility, improved strengths with adhesion qualities with compressibility to improve upon existing technology. Furthermore, the discovery of the molded article encountered challenges in combining unlike polymers to compatibilize in a compound with synergistic fillers like cellulose fibers of a particular shape that will offer unique attributes and comply with regulations associated with particular markets. Moreover, the polymers, consisting of polyamide and polypropylene, will need to be compatibilized before melt blending with the cellulose fiber to produce a pelletized compound for 3 D printing. Consequently, the 3 D printing process will produce a molded article from filament or powders from these pellets, whereby, the cellulose fiber particles are coated in the thermoplastic matrix and finely dispersed. Furthermore, these cellulose fiber particles are not oriented to achieve a molded article having low shrink of x, y and z.

Because these cellulose fiber particles are not compressed and exposed in the layering process of the molded article, they can trap moisture and gases, which can helping create a slightly positive charge. The moisture from the hydrophilic ingredients will include a water molecule, have two hydrogen atoms, a slightly positive charge and the oxygen atoms attracts electrons, giving it a slightly negative charge. Furthermore, the positive and negative ends make the moisture, or water molecule like a magnet (dipole), which keeps water molecules together and makes the surface of a water drop slightly elastic. Consequently, a layering of the cellulose fiber particles, having both negative and positive charge, has promoted a unique surface energy beyond the 70 dynes/cm for coatings of nickel and copper. The ability to metal coat this 3 D printed article having cellulose fiber particles, i.e. fused deposition modeling (FDM) can provide novel sensory feedback for diagnostics. Moreover, the combination of the 3 D printing process and a biopolymer material composition having non orientation of cellulose fiber particles coated with a combination of hydrophilic and hydrophobic polymers can produce improved multidimensional articles referenced by the inventor to include filters, i.e. membrane, cartridge, and or backing.

### BRIEF DESCRIPTION OF DRAWING

FIG. 1. is a top view of a printed part having cellulose fiber particles not compressed and layered with a region charged by a gas and coated with metal.

### DESCRIPTION OF RELATED ART

Molded articles have used cellulose with polyamide, polypropylene and cellulose fibers. When utilizing these fibers with a thermoplastic polymer, there has been focus on achieving maximum tensile strength or flexural modulus with light weighting for producing large parts. The automotive industry has been diligent in exploring this composition of cellulose, PA/PP blends. In 2015, Society of Plastic Engineers Awarded Ford Motor Company Engineers by publishing Selective Dispersion and compatibalizing effect of Cellulose filler with PA/PP Blends. In this paper, Ford Motor Company advanced manufacturing engineers molded cellulose with thermoplastic polymers in a process that required compression of the fibers in the thermoplastic matrix like injection or compression molding above 10,000 psi. Consequently, the molded parts that were produced had agglomeration and inconsistent batch mixing creating dispersion issues of the fibers. Further experimentation to create molded articles with cellulose, polyamide, polypropylene blends since 2009 had been performed by Joyce, where he has several U.S. patents to include U.S. Pat. No. 7,994,241 and Joyce et. al U.S. Pat. No. 8,546,470, U.S. Pat. No. 9,109,188. The inventor has now discovered through a process of experimentation to mold parts without out pressure that cellulose can be dispersed in fine particles without agglomeration in a thermoplastic alloy composition having to produce layers in a semi solid state. These layers will have cellulose fiber particles that require improved adhesion will produce a buoyant molded article.

Printed articles having a biopolymer material composition, natural fibers, cellulose and thermoplastic, requires a composition that conforms with the process. In this case, 3 D printing has very little if any associated pressures to apply a biopolymer material to a cavity. Consequently, the adhesion between the natural fiber and thermoplastic has to be exceptional for creating the 3 D printed article. The inventor has chosen an organic compound, i.e. cellulose fiber of a high glass transition temperature, low moisture and high purity to alleviate some of the challenges like adhesion mentioned above. Other identified compositions of earlier art that contain cellulose fiber, polyamide and polypropylene have not required the necessary adhesion for layering in a process under compression that require these cellulose fiber particles to finely disperse in a semi solid state to create this buoyant molded article.

There are medical filters that are radial compressible and expandable, i.e. Victor Farmiga published patent application Extended duration medical filter with bio-absorbable barbs in 2008. Farmiga identifies polymers and bio materials such as PLA (poly lactic acid) in an extrusion process to create the molded tubular article where by the cage is compressible in a solid state and the barbs are created to stabilize the filter in a artery. Farmiga does have challenges to create the medical filter, due to the tubular extrusion construction that require a secondary process to create the tiny barbs. Farmiga doesn't declare a molded article constructed of layers consisting of cellulose fiber particles in multiple thermoplastic polymers. Furthermore, there is no declaration of cellulose fiber particles being compressible in a semi solid state to create a molded article having a specific gravity less than 1.0 g/cm3. Furthermore, any type of medical filter having membranes, have graded permeability based on their pore sizes and level of uniformity. The materials used for membranes are of an existing polymer resin such as PLA, ABS, Nylon. Cellulose esters, plastics, and PTFE are generally hydrophobic, which obviously hinders filtration of aqueous solutions. Water can be forced through the hydrophobic filters, but the pressure required to force water through pores smaller than 1 micron becomes prohibiting high, and the risk of uneven wetting occurs. Thus, most membranes are treated with wetting agents, such as glycerol, which allows water to flow freely. Most laboratory and medical applications call for membranes that are generally less than 0.1 mm thick with a precise pore diameter. Modern membranes are manufactured under rigid parameters to deliver high porosity and support high flow rates. Two broad applications for filters are the production of particle free solutions and particle capture. These filters are commonly used for diagnostic, assays like electrophoresis, cytology of fluids. Other systems where membranes are found include oxygen detectors, pH and Ion electrodes.

Since membrane filtration deals with very small particles, electric charge can be greatly affect filter efficiency and performance. When wet, most filters have a negative charge. At neutral pH, most cells, viruses and macromolecules also exhibit a negative charge. The repulsion of like charges can overcome electrostatic attraction and van der Waals forces need to absorb small particles into the membrane, leading to less than optimal performance. This invention can utilize both charges and perform exceptionally well under an aqueous and gaseous state. Plus, the molded article can be sterilized without a substantial reduction in properties.

### BRIEF SUMMARY OF THE PRESENT INVENTION

Many printed articles such as medical filters use existing materials like titanium, cellulose acetate, cellulose nitrate and resins such as polyamide and polypropylene. There are very few filters that are constructed of plastic natural fiber composite materials. The inventive molded filters will include ingredients that consist of hydrophilic and hydrophobic thermoplastic resins and an organic compound, i.e. cellulose fiber, having additional additives as required like lubricants, couplings, minerals, compatibilizer, pigments, antimicrobials to produce a cellulose thermoplastic alloy composition. By means of compounding the ingredients in an extrusion process below 410 F or 210 C, the process will produce at a minimum the composition consisting of cellulose, plus, water and a plastic alloy that has a high heat polyamide and or PET and one or more polyolefins such a polypropylene. This composition has shown that after molding with no or minimal pressures, i.e. 3 D printing tiny cellulose fiber particles are exposed at the surface having a thermoplastic coating. Furthermore, these tiny fiber particles are not oriented, unlike the extrusion process that created the 3 D filament where a larger percentage of fibers are oriented. The composition in the 3 D printing process will help create new filters with improved sterilization, better strength and impact properties with porosity, absorbency qualities and promote a positive electrical charges associated with many filter applications in the medical field.

The cellulose fibers compounded with the polyamide and polyolefin, which we can describe as a non polar and polar polymers having different melt temperature, has a small percentage of water which has a sticky quality that comes from its molecular structure. The oxygen atom attracts electrons, giving it a slightly negative charge. The two hydrogen atoms have a slightly positive charge. The positive and negative ends make the water molecule like a magnet (dipole), which keeps water molecules together and makes the surface of a water drop slightly elastic. It also means that water molecules will bond easily with molecules with an opposite charge that come nearby. Furthermore, the cellulose thermoplastic alloy composition has an affinity to hold moisture but not store aqueous solutions. The coatings processes that are preferred include gases, though aqueous solutions can be applied, have shown to be compatible and adhere to metal coatings. Non metal coatings can also be used to coat the molded article described herein, to help reduce inflammation for implantable medical devices.

Unlike a thermoplastic starch or poly lactic acid which stores liquids or energy, cellulose fibers do not store energy and are coated with a hydrophilic and hydrophobic polymers, referred to as “cellulose fiber particles” in this molded article. These cellulose fiber particles are not oriented in the 3 D printing process, producing a molded article that has compressibility and buoyancy. Based on previous methods and materials to produce printed articles, i.e. medical filters there lacks a capability for trapping particles with two polymers of different polarity and a organic compound having cellulose fiber particles with a specific gravity below 1.0 g/cm3. The 3 D printing process will have no or minimal pressures besides gravity, and can produce articles such as membranes and cartridges for filters. Furthermore, the inventive 3 D printed molded article will comprise of cellulose fiber particles in a thermoplastic alloy composition at densities below the specific gravity of 1.0 g/cm3 and measure by ISO and ASTM standard testing methods greater than 1.05 g/cm3. There are limitations in a 3 D printing process Fused Deposition Modeling (FDM) whereby a membrane pore size cannot be below 50 micron. Because the composition is compressible in a semi solid state, using pressure and heated platens, the molded article reach an improved pore size of less that 25 micron, a 50% improvement with increased accuracy versus previous methods. Secondary process methods can also be improved and utilized for assisting in further part development.

### DETAILED DESCRIPTION OF INVENTION

The biopolymer material composition used to create the molded articles will have organic compound with hydrophilic and hydrophobic polymers for melt blending to form a single composition. Moreover, it is preferred that the single composition will comprise of two separate compounding processes for producing a thermoplastic alloy compound with the organic compound into one composition. This composition will be a cellulose thermoplastic alloy compound that will can be formed into a pellet of 3 mm or less, processed further into a filament or powder for 3 D printing. In a preferred 3 or 4 dimensional printing process, or any additional printing axis to include a 5 or 6 axis, cellulose fiber particles are evident having contributed to a layered printing process, that will help create a molded article to exhibit buoyancy and be compressible in a semi solid state.

In order to produce a composition containing cellulose fiber with high heat and polyolefin polymers, it is required to first melt blend and compatibilize the polymers at processing temperatures at approximately 240 C to 280 C. Then melt blend the compound with the cellulose fibers downstream where temperatures are approximately 180 to 200 C which is less than the glass transition temperatures of the cellulose fibers. The cellulose fiber has a minor decomposition reaction take place at 266° C. and the major decomposition at 315° C. with a glass transition point between 220 to 250 C, having a 5 to 7% moisture content. If there is need to utilize additives with cellulose fibers that require similar low processing temperatures in the 180 to 200 C like boric acid, the boric acid can be blended downstream with the cellulose fiber, so that, degradation will be minimized. Furthermore, at the point where the thermoplastic alloy will be melt blended with the cellulose fiber, there will be some removal of moisture downstream, but some water and or moisture will react with the ingredients to help with the stickiness and molecular structure. Consequently, the molded article will have some absorption of moisture, however, the buoyancy can persist for an extended period of time due to the cellulose fiber particles unlike other biomaterials like PLA (Poly Lactic Acid).

The ingredients to make the molded article from a 3 or 4 dimensional printing process will have cellulose fiber particles that are layered and not oriented having a very unique surface texture. The organic compound, i.e. cellulose fiber is a preferred filler, but could include microcrystalline cellulose, in the molded article to produce these cellulose fiber particles with the thermoplastic alloy, have a mesh size below 80 or approximately 175 micron with a preferred mesh size less than 120 or 125 micron and a most preferred particle size distribution size retained at 65% maximum with 35 micron. An aspect ratio is preferably below 15 to 1 and more preferably in the 5 to 1 range and purity no less than 99%. The polyamide can be of various strains to include an amorphous and or crystalline. i.e. Nylon 6, 6/6, 11, 12. The polypropylene can be a co or homo polymer, high crystalline with a high melt flow. Both of these polymers will need to be compatibilized and have adhesion between the fibers. The inventor prefers a modified elastomer to adhere fibers to polymers. These fibers will have minimal shearing in the compounding process, as well as, in the layering process for printing the molded article.

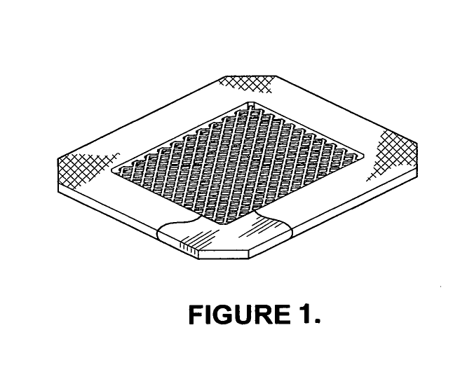

The inventor has found that by printing this composition in a layered process with little or no compressible forces, a reduction of 10 to 20% by weight was discovered, and can be as high as 30%, which can produce a buyout molded article, having a unique surface texture. Furthermore, the loose compaction of the composition in a layered process, enabled the cellulose fiber particles to help trap gas and aqueous solutions. The 3 D printing process produced this part below. FIG. 1 having a specific gravity less than 1.0 g/cm3, though the cellulose thermoplastic alloy composition consisted of at least 12% of cellulose fiber and tested under ISO standard methods had a specific gravity of 1.05 g/cm3. The compressible nature of the composition is required to produce articles such as filters because of the limitations in a 3 D printing process caused fused deposition modeling (FDM). It has been determined that a cellulose thermoplastic alloy composition will have a high degree of compressibility versus other polymeric or filled resins, whereby unique articles such as filters, having pores can be produced with very small dimensions 100 to 50 micron. The FIG. 1 part was produced through a 3 D printing FDM process utilizing a Prusa i3v printer, whereby the process temperatures were 240 C., a nozzle size of 0.8 mm, a print speed of 4 mm/second and a heated bed at 80 C. A software program called Marlin with slic3r.

In any article, especially a filter membrane, a predictable micron size of a pore can be limited in FDM (fused deposition modeling) to only 50 micron. This micron size of the pores are not conducive to achieving the necessary functionality in some applications. Many filter applications in the medical market require 1 micron or smaller and can be reusable if necessary. A certain amount of compressibility of the cellulose thermoplastic composition will be required to achieve the 1 micron pore size. Depending on the amount of cellulose and moisture in the composition, compress ability will enable one to create a smaller pore size than the capability of the 3 D printer. In some cases, secondary process, either a vertical or horizontal press having two heated platens is required. Some multidimensional printers, an SLS (Selective Laser Sintering) is used for production to achieve improved resolution of pore size. In either case, the molded article will utilize cellulose fiber particles for creating a pore size less than the printed article. To decrease the mesh or micron size of a membrane or pore, the platens require heat between 290 F to 320 F to make the cellulose composition a semi solid for compression. The pressure of the vertical press requires a force between 200-600 lbs per sq. inch to achieve a reduction of 30 to 50% in a single pore diameter. Consequently, if the membrane is attached to a frame both structures will grow in size. Not only will the pore size decrease in size but the frame associated with the membrane will increase in diameter by similar size. In order to achieve the 1 micron resolution multiple layers will need to be constructed, set on top of one another, then heated and pressurized.

The process for printing the filter will require either a cellulose thermoplastic alloy filament or powder. The filament will be either a 1.75 mm or 3.0 mm size with orientation of cellulose fibers in the thermoplastic matrix. It has been observed that the filament can have exceptional strength length wise but not as much cross section. The inventor can use compounded pellets and prefers to produce powders to make these filaments. The powder comprising of an organic compound with a thermoplastic alloy composition will be no bigger than 10 mesh. This invention requires that the organic compound, is preferably cellulose fibers having more than 90% purity and more preferably close to 100%.

The inventor had discovered that the printed article, FIG. 1 will be buoyant due to the layering process that includes tiny cellulose particles with thermoplastic polymers that are non oriented, consequently trapping moisture or gas. The filament that produced the molded article had a smooth texture was processed in high temperatures above 220 C, beyond the degradation temperatures of the organic compound or cellulose fibers. It was also observed that this 3 D printed part, which had little or no pressure, can process the composition with a 0.4 mm nozzle tip with a 240 C processing temperature. Furthermore, the composition showed no visible degradation of the cellulose fiber particles and helped create a surface texture that was not smooth and shinny.

The inventor has identified the medical filter as an example of an inventive molded article having buoyancy and compressibility created through a printing process which layers cellulose fiber particles. A membrane and cartridge in the filter can use the same composition to help reduce field failures because of two or more materials having the necessary adhesion of backings like polypropylene or polyethylene in an aqueous solutions. Depending on the molded filter requirements, a 3 D printed part made with the cellulose thermoplastic alloy composition described herein, can be inserted into a tool, to be insert molded, i.e. micro molded, to add improved compatibility or adhesion between components. This molded article can replace existing paper filters with improved strength and or improved structure to support in spillover. For instance, the composition will improve the adhesion with PTFE versus existing backer resins such as polypropylene or polyethylene that has no polarity or adhesion qualities without a secondary process.

1. A molded article produced from a 3 or 4 dimensional printing process, that may include a 5 or 6 axis, comprising of an organic compound with hydrophilic and hydrophobic polymers, wherein, said printed article has cellulose fiber particles that are layered in a semi solid state for buoyancy.

2. The molded article as claimed in claim 1 wherein said article shall trap fluids and gases for metal coating.

3. The molded article as claimed in claim 1 wherein said article contains polyamide and polypropylene polymers.

4. The molded article as claimed in claim 1 wherein said article contains cellulose fibers and thermoplastic polymers that are compatibilized for production of one composition for.

5. A molded article produced from a 3 or 4 dimensional printing process comprising of cellulose fiber with thermoplastic polymers having a specific gravity less than 1.0 g/cm3 and measured with ASTM or ISO methods greater than 1.05 g/cm3.

6. The molded article as claimed in claim 5 wherein said article comprises of a cellulose fiber having a glass transition point between 200 to 240 C and has over 90% purity.

7. The molded article as claimed in claim 6 wherein said article contains cellulose fiber with semi crystalline and or amorphous polymers.
